(19) United States
(12) Patent Application Publication
(10) Pub. No.: US 2005/0101925 A1
(43) Pub. Date: May 12, 2005

(54) Disposable diaper underpad

(76) Inventor: Evelyn Marie Oliver, Southfield, MI (US)

Correspondence Address: Evelyn Marie Oliver, 29668 Farmbrook Villa Ct., Southfield, MI, 48034, US

(21) Appl. No.: 10/703,612

(22) Filed: Nov. 7, 2003

(51) Int. Cl. A61F 13/15; A61F 13/20

(52) U.S. Cl. 604/358

(57) ABSTRACT

The size is 23″×36″ and/or 58 cm×91 cm. Soft and smooth in texture as well as flexible. The area touching the surface has a plastic back lining with two adhesive lines of equal length and width, vertically placed for purpose of stability. The area facing upward is made of 1 inch thick super absorbent polymer with odor control.

A. Non-Flushable And Disposable Underpad

B. Size is 23″×36″ and/or 58 cm×91 cm

C. 2 inches of ultra extra absorbency diaper padding materials that is placed on a plastic lining and serve as the outer lining while the diaper is the inner section that is utilized.

D. Two adhesive lines are placed equal to length And/or width of product that is located on the Outer plastic lining for purpose of stability.

A. This product can be used to meet the need of Adults with incontinence. This product can be Spread Out over bed or chair where a person is Expected to lay or sit buttocks area of their Body. If an accident occur this product has a Thick enough padding to absorb urine without Soiling the bed and/or chair.

B. This product can be used to pad a wheel chair Just in case a urination accident occur.

C. This product can be spread over a surface While under a baby being washed up or diaper Change just in case of urination before re-Diapering.

D. Due to this product's disposal feature, flexibility And absorbent quality it can be useful in Hospitals.

1. I claim this invention as a useful, sizable, disposable diaper underpad that absorbs moisture and supports dryness to individuals whenever it is placed on a surface, under buttocks area of the body during incontinence situations and/or excretory functions: This Disposable Diaper Underpad is flexible with a plastic backing, an absorbent front like a diaper, but flat like a padding. It operates by spreading out over a bed, chair, wheel chair or any surface to absorb excretory functions and support dryness. For example, spread out this Disposable Diaper Underpad on the seat of a wheel chair for support, beds at home or hospitals for purpose of excretory functions, medical testing or infant diaper changing.
